(19) United States
(12) Patent Application Publication
(10) Pub. No.: US 2007/0017505 A1
(43) Pub. Date: Jan. 25, 2007

(54) Dispensing device and method

(76) Inventors: Brian A. Lipp, Columbus, OH (US); David Cline, Dublin, OH (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 11/485,787

(22) Filed: Jul. 13, 2006

Related U.S. Application Data
(60) Provisional application No. 60/699,932, filed on Jul. 15, 2005.

(51) Int. Cl. A61M 11/00

(52) U.S. Cl. 128/200.16

(57) ABSTRACT

Methods and devices for dispensing an aerosolized liquids that generate an electric field proximate an outlet of a liquid supplier to cause liquid issuing from the outlet to be aerosolized for dispensing, and regulating an electrical characteristic, such as voltage, for generating the electric field based on a detected electrical characteristic, e.g., current drawn, of the electric field and/or based on an environmental sensor, to compensate, for example, for adverse effects of relative humidity in the aerosolization process. Such methods and devices are suitable for use as, for example, an inhaler for dispensing therapeutic liquids to a patient's lungs, spraying paint, crops or other liquids over a surface area.

### CROSS REFERENCE TO RELATED APPLICATION

This application claims priority from U.S. Provisional Application No. 60/699,932 filed on Jul. 15, 2005. The above mentioned application is incorporated herein by reference.

### FIELD OF THE INVENTION

The invention relates generally to devices and methods for spraying liquids and specifically to devices and methods that electrostaticly aerosolize liquids for spraying.

### BACKGROUND OF THE INVENTION

Devices and methods for forming fine sprays by particular electrostatic techniques are known. For example, U.S. Pat. No. 4,962,885 to Coffee, incorporated by reference herein, describes a process and apparatus to form a fine spray of electrostaticly charged droplets. More specifically, the process and apparatus comprise a conductive nozzle charged to a potential of the order of 1-20,000 volts, closely adjacent to a grounded electrode. A corresponding electric field produced between the nozzle and the grounded electrode is sufficiently intense to atomize liquid delivered to the nozzle, and thereby produce a supply of fine charged liquid droplets. However, the field is not so intense as to cause corona discharge, with resulting high current consumption. Advantageous uses of such liquid dispenser process and apparatus include sprayers for paint and/or spraying of crops. Aerosolization of liquids using electric fields is often referred to as electrostatic aerosolization of the liquid.

More recently, there has been a recognition that such spraying devices are extremely useful for producing and delivering aerosols of therapeutic products for inhalation by patients. In one particular example, described in U.S. Pat. No. 6,302,331 to Dvorsky et al. incorporated by reference herein, fluid is delivered to a nozzle that is maintained at high electric potential relative to a proximate electrode to cause aerosolization of the fluid with the fluid emerging from the nozzle in the form of, for example, a so-called Taylor cone. One type of nozzle used in such devices is a capillary tube that is capable of conducting electricity. An electric potential is placed on the capillary tube which charges the fluid contents such that as the fluid emerges from the tip or end of the capillary tube in a manner to form the Taylor cone.

The Taylor cone shape of the fluid before it is dispensed results from a balance of the forces of electric charge on the fluid and the fluid's own surface tension. Desirably, the charge on the fluid overcomes the surface tension and at the tip of the Taylor cone, a thin jet of fluid forms and subsequently and rapidly separates a short distance beyond the tip into an aerosol. Studies have shown that this aerosol (often described as a soft cloud) has a uniform droplet size and a high velocity leaving the tip but that it quickly decelerates to a very low velocity a short distance beyond the tip.

Electrostatic sprayers produce charged droplets at the tip of the nozzle. Depending on the use, these charged droplets can be partially or fully neutralized (with a reference or discharge electrode in the sprayer device) or not. The typical applications for an electrostatic sprayer, without means for discharging or means for partially discharging an aerosol would include a paint sprayer or insecticide sprayer. These types of sprayers may be preferred since the aerosol would have a residual electric charge as it leaves the sprayer so that the droplets would be attracted to and tightly adhere to the surface being coated. Under certain circumstances (i.e., delivery of some therapeutic aerosols), it may be preferred that the aerosol be completely electrically neutralized.

Moreover, electrostatic-type inhalers, in which the charge on the droplets is typically neutralized, have demonstrated advantages over more conventional metered dose inhalers (MDI) including producing more uniform droplets, enabling the patient to inhale the formed aerosol liquid or mist with normal aspiration, producing higher dosage efficiencies, and providing more reproducible doses.

It is often advantageous and/or important to consistently reproduce an aerosolized liquid having a particular physical characteristic, e.g., droplet size, size distribution, rate of aerosolization, or plume angle for maintaining a consistent therapeutic product dosage or for a stable applications of a liquid over crops or surfaces to be painted or other non-medicinal applications. However, variations in environmental factors, such as humidity, temperature, or barometric pressure due to climate variations, changes in altitude, or otherwise, or production variations in the dispenser configuration including nozzle geometry, often make it difficult to consistently and repeatedly produce the desired physical characteristic(s) in the aerosolized liquid. As a consequence, devices that can deliver consistent aerosol properties under extremes of operating conditions have not been available. Such devices had to be operated within limited humidity, temperature or altitude ranges in order to consistently produce the aerosolized liquid with the desired physical characteristics. In reality, changes in properties of the air between the electrodes can lead to inconsistent performance with respect to droplet production. In addition, costly rigid manufacturing variances and tolerances are required for manufacturing such devices. Small variations in nozzle geometry such as electrode positions have adverse consequences in the formation of aerosolized liquids consistently having desired characteristics. Accordingly, it is desirable to develop a method for aerosolizing a liquid that is highly robust and not influenced by changes in operating conditions such as environmental parameters or small changes in device geometry.

Thus, improved dispensing devices and methods are desired to overcome the requirements for rigid manufacturing tolerances and operation of electrostatic spraying devices within limited environmental ranges.

### SUMMARY OF THE INVENTION

This invention is based on the discovery that it is possible to compensate for variations in operating conditions such as, for example, different humidity, temperature and barometric pressure, to maintain a desired characteristic of an aerosolized liquid by regulating an electrical characteristic such as, for example, voltage, used for generating the electric field which is used to produce the liquid droplets. The value of the particular electrical characteristic being regulated can be calculated from measurements made by an environmental sensor located in the proximity of the electrodes. In accordance with an alternative embodiment of the invention, it has been discovered that it is also possible to determine the value for the particular electrical characteristic being regulated based on a detected different electrical parameter such as, for example, current, in the circuit used to generate the desired electric field.

Thus, the invention is directed to methods and devices for generating an electric field proximate to an outlet of a liquid supplier to cause liquid issuing from the outlet to be aerosolized and regulating an electrical characteristic, e.g., voltage, for generating the electric field based on a detected parameter of the operating environment or circuit used to generate the electric field to compensate for differing operating conditions. The detected parameters may be an electrical characteristic of circuit generating the electrical field, e.g., current drawn, or measurements from environmental sensors.

In accordance with one embodiment of the invention, it is possible to compensate for adverse effects of changing relative humidity and other environmental conditions in the aerosolization process by regulating the voltage used for the electric field generation. In accordance with one example of such embodiment, the voltage is regulated to (1) provide a substantially constant voltage, such as, for example, in the range of 10 kV and 12 kV for generation of the electric field when the current drawn by such electric field generation is within a first range such as, for example, between 0 μA and 10 μA; and (2) provide a substantially constant power wherein the voltage is adjusted based on the current drawn to maintain such substantially constant power when the drawn current is greater than 10 μA. In such an example, the characteristic of droplet size formed in the aerosolized liquid is in a desired range such as, for example, between 0.1 and 6 microns despite such formation being subjected to a broader range of environmental conditions than is achievable with present electrostatic aerosolization liquid dispensers.

The present invention is also useable for aerosolizing different liquids having different electrical properties by determining, empirically or otherwise, the necessary electrical characteristic profile for voltage and current regulation required for maintaining a substantially constant characteristic of an aerosolized liquid over a broad range of operating conditions. In accordance with the present invention, a liquid dispenser effectively maintains a desired physical characteristic in the aerosolization of a liquid by compensating for a larger range of environmental conditions than present liquid dispensers including compensating for manufacturing variations that may occur in mass production of such dispensers.

Suitable applications of the invention include, for example, to spraying crops, applying paint or delivery of therapeutic liquids in an inhaler to a patient's lungs.

### BRIEF DESCRIPTION OF THE DRAWINGS

The accompanying drawings incorporated in and forming part of the specification illustrate several aspects of the invention, and together with the description serve to explain the principles of the invention. In the drawings:

FIG. 1 is a schematic diagram of an exemplary aerosolized liquid dispenser in accordance with the invention;

FIG. 2 is a schematic diagram of an exemplary regulated power supply useable in the aerosolized liquid dispenser of FIG. 1;

FIG. 3 is a chart depicting an exemplary voltage-current function curves for illustrating the operation of the regulated power supply of FIG. 2; and

FIG. 4 is exemplary alternative voltage-current function curves to that of FIG. 3 for illustrating the operation of the regulated power supply of FIG. 2; and

FIG. 5 is an alternative embodiment of the regulated power supply of FIG. 2.

### DETAILED DESCRIPTION

The invention relates to methods and devices for electrostaticly aerosolizing liquid for the purpose of spraying. In particular, the invention provides the capability to repeatedly form such aerosolized liquids having a substantially consistent particular characteristic in a desired range despite being subjected to a variety of environmental conditions such as, for example, differences in humidity, temperature, barometric pressure or manufacturing variations in the sprayer configuration. Suitable applications of the invention include, for example, spraying crops, applying paint, or delivering liquids having therapeutic properties by way of an inhaler to a patient's lungs.

Although the following description primarily focuses on an exemplary pulmonary delivery device (inhaler) implementation of the invention, it should be readily understood that such teachings apply to sprayers in other applications. Other suitable applications of the invention include, for example, to spray crops, paint or to generally coat surface areas with other liquids. The description further teaches different aspects of the invention by electrohydrodynamic (EHD) aerosolization of the therapeutic fluid with the aerosolized fluid emerging from a nozzle in the form of a so-called Taylor cone.

Liquids suitable for aerosolization by EHD spraying generally are characterized by particular electrical and physical properties. For example, without limiting the scope of the invention, liquids having the following electrical and physical characteristics permit optimum performance by the device and method to generate a clinically relevant dose of respirable particles within a few seconds: (1) Liquid surface tension typically in the range of about 15-50 dynes/cm, preferably about 20-35 dynes/cm, and more preferably about 22-33 dynes/cm; (2) Liquid resistivity typically greater than about 200 ohm-meters, preferably greater than about 250 ohm-meters, and more preferably greater than about 400 ohm-meters (e.g., 1200 ohm-meters); (3) Relative electrical permittivity typically less than about 65, preferably less than about 45; and (4) Liquid viscosity typically less than about 100 centipoise, preferably less than about 50 centipoise (e.g., 1 centipoise). Although the above combination of characteristics allows optimum performance, it may be possible to effectively spray liquids with one or more characteristics outside these typical values using the device and method of the invention. For example, certain sprayer nozzle configurations or electrode placement may allow effective spraying of less resistive (more conductive) liquids.

Generally, therapeutic agents dissolved in ethanol are good candidates for EHD spraying because the ethanol base has a low surface tension and is nonconductive. Ethanol also is an antimicrobial agent, which reduces the growth of microbes within the drug formulation and on the housing surfaces. Other liquids and solvents for therapeutic agents also may be delivered using the device and method of the invention. The liquids may consist of drugs, or solutions or suspensions of drugs in compatible solvents.

As described above, the EHD apparatus aerosolizes the liquid by causing the liquid to flow over a region of high electric field strength, which imparts a net electric charge to the liquid. In the present invention, the region of high electric field strength sometimes is provided by a negatively charged electrode within the spray nozzle. The negative charge tends to remain on the surface of the liquid such that, as the liquid exits the nozzle, the repelling force of the surface charge balances against the surface tension of the liquid. The electrical force exerted on the liquid surface overcomes the surface tension, generating a thin jet of liquid. This jet breaks into droplets of more or less uniform size, which collectively form a cloud. In another embodiment, the electrode is grounded while the discharge electrode is positively charged (at, for example, twice the voltage), or the nozzle electrode can be positive. In any case, a strong electric field is required.

The device is configurable to produce aerosolized particles of respirable size. Preferably, such respirable droplets have a diameter of less than or equal to about 6 microns, and more preferably, in the range of about 1-5 microns, for deep lung administration. In formulations intended for deep-lung deposition, it is preferable that at least about 80% of the particles have a diameter of less than or equal to about 5 microns for effective deep lung administration of the therapeutic agent. The aerosolized droplets are substantially the same size and have near zero velocity as they exit the device.

The range of volumes to be delivered to the pulmonary system is dependent on the specific drug formulation. Typical volumes are in the range of 0.1-100 μL. Ideally, the dose should be delivered to the patient during a single inspiration, although delivery during two or more inspirations may be acceptable under particular conditions. To achieve this, the device generally must be capable of aerosolizing about 0.1-50 μL, and particularly about 10-50 μL, of liquid in about 1.5-2.0 seconds. Delivery efficiency is also a major consideration for the pulmonary delivery device so liquid deposition on the surfaces of the device itself should be minimal. Optimally, 70% or more of the aerosolized volume should be available to the user.

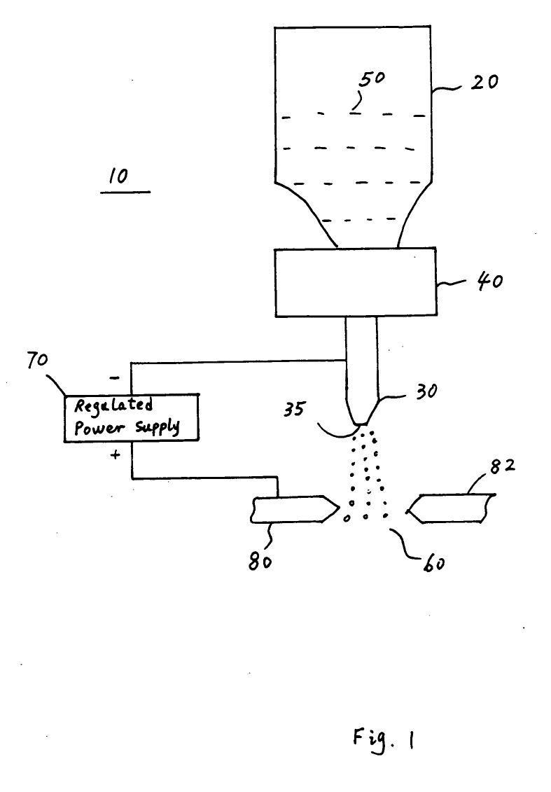

In the Drawings, like reference numerals represent like components throughout the figures. FIG. 1 depicts a schematic diagram of an exemplary pulmonary delivery device 10, i.e., inhaler, according to one embodiment of the invention. Such a device may include a housing (not shown) sized to enable handheld or table-top operation. Moreover, the inhaler 10 may preferably be cordless, portable and provide consistent multiple daily doses over a period of days or weeks without refilling or user intervention. The inhaler 10 includes a containment vessel 20 connected to an nozzle 30 via pump and valve mechanism 40 for dispensing a particular quantity of liquid 50 of, for example, 0.1 μL to 100 μL, contained in the vessel 20 for aerosolization from outlet 60.

A regulated power supply 70 is electrically coupled to the nozzle 30 and discharge electrodes 80 and 82. The discharge electrodes 80 and 82 are positioned proximate to the nozzle 30 to create a corresponding electric field such that liquid emanating from a tip 35 of the nozzle 30 is aerosolized for discharge from outlet 60. The electric field is created by the power supply 70 by producing a sufficient voltage potential ΔV between the electrodes 80 and 82 relative to the nozzle 30. Exemplary ranges for the voltage potential ΔV are 8 KV to 20 KV, more preferably between 8 KV to 12 KV and most preferably 11 KV.

The liquid 50 to be aerosolized is held in the containment vessel 20 that stores and maintains the integrity of the therapeutic liquid. The containment vessel 20 may take the form of a holder for a drug enclosed in single dose units, a plurality of sealed chambers each holding a single dose of the drug, or a vial for enclosing a bulk supply of the drug to be aerosolized. Bulk dosing generally is preferred for economic reasons except for liquids that lack stability in air, such as protein-based therapeutic agents. The containment vessel 20 preferably is physically and chemically compatible with the therapeutic liquid including both solutions and suspensions and is liquid and airtight. The containment vessel 20 may be treated to give it antimicrobial properties to preserve the purity of the liquid contained in the containment vessel 20. Suitable containment vessels are further described in, for example, U.S. patent application Ser. No. 0/187,477, which is incorporated by reference herein.

The pump and valve mechanism 40 provides a desired amount of the liquid from the vessel 20 to the nozzle 30 at a desired pressure or volumetric flow rate. However, the specific configuration chosen for the pump and valve mechanism 40 to perform such function is not critical to practicing the invention. Suitable configurations for the pump and valve mechanism 40 are described in U.S. Pat. Nos. 6,368,079 and 6,827,559, which are incorporated by reference herein. Additional pump configurations for the pump 40 are also disclosed in U.S. Pat. No. 4,634,057, which is likewise incorporated by reference herein. The containment vessel 20 alone, or in combination with the pump and valve mechanism 40, provide a liquid supplier for aerosolization of liquids maintained by the containment vessel 20.

Suitable nozzle configurations for the nozzle 30 include, for example, those nozzle configurations described in U.S. Pat. Nos. 6,397,838, and 6,302,331 and U.S. patent application Publication No. 2004/0195403 which are incorporated by reference herein.

In the depicted embodiment of the invention in FIG. 1, the nozzle 30 and electrodes 80 and 82 operate as an electric field generator powered by the power supply 70. The depicted positioning of the electrodes 80 and 82 relative to the nozzle 30 in FIG. 1 is such that an electric field would be produced between the tip 35 of the nozzle 30 and the electrodes 80 and 82. However, it is possible to alternatively position the electrodes 80 and 82 adjacent to or behind the nozzle tip 35 (in a direction away from the outlet 60) for creating the electric field at or behind the nozzle tip 35. Moreover, it is also possible to employ a single electrode instead of the two electrodes 80 and 82 in accordance with the invention. In a similar manner, it is further possible to employ a larger number of electrodes to create the required electric field.

FIG. 1 depicts the use of two electrodes 80 and 82 relative to the electrically conductive nozzle 30 for illustration purposes only. It is advantageous in accordance with the invention to have an electric field sufficiently large for effective and efficient aerosolization of the issuing liquid. To this end, it is possible to employ a larger number of corresponding electrodes or a ring electrode proximate to the electrically conductive nozzle 30. In addition, it is likewise possible to employ electrically conductive strips or rings formed within the nozzle 30 for providing its portion of the electric field generator configuration. Exemplary alternative electric field generator configurations are useable in accordance with the invention including, for example, the configurations described in U.S. Pat. No. 6,302,331 and U.S. patent application Ser. No. 10/375,957, which are incorporated by reference herein.

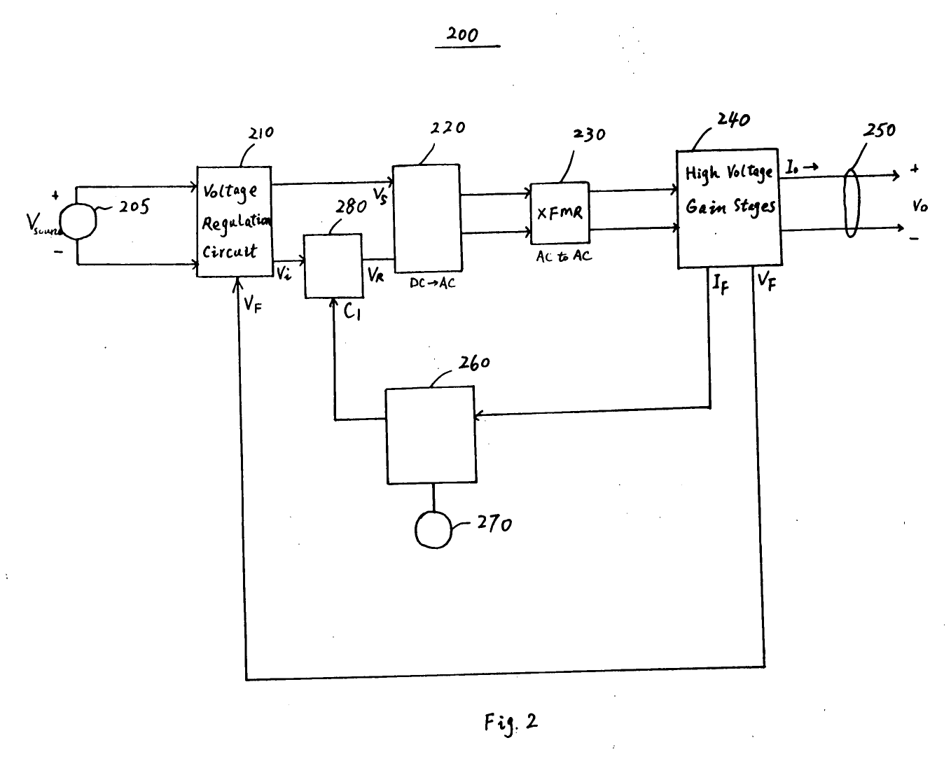

One exemplary circuit 200 usable for the power supply 70 of FIG. 1 is depicted in FIG. 2. The power supply circuit 200 includes a power source 205, such as a battery, that provides a voltage V_{SOURCE} coupled to a voltage regulation circuit 210 that is electrically connected to provide a voltage Vᵢ to a current control circuit 280 and a voltage Vₛ to a switching circuit 220. The voltage V_{S} is based on the voltage V_{SOURCE} provided to the voltage regulation circuit 210. An output V_{R} of the current control circuit 280 is electrically coupled to the switching circuit 220. The output of the switching circuit 220 is connected to a transformer 230 which in turn, is connected to high voltage multiplier stages 240 having electrical outputs 250. The outputs 250 would be electrically connected as depicted to the electrodes 80 and 82 (and/or electrically conductive nozzle 30) in FIG. 1.

Referring again to FIG. 2, the high voltage multiplier stages 240 further produces feedback signals V_{F} and I_{F} indicative of voltage V_{O} and current I_{O} at the outputs 250, respectively. The signals V_{F} and I_{F} are provided to a controller 260 which produces voltage control signal C₁, that control the operation of the current regulator circuit 280. In addition, the signal V_{F} is also provided back to the voltage regulation circuit 210. It is possible to employ readily available high voltage generator parts for the respective components 210, 220, 230 and 240, such as, for example, those available from HiTek Power Corp of Santee, Calif. Moreover, it should be readily understood by one skilled in the art that is possible for the controller 260 to be implemented as an analog controller circuit, or a digital circuit such as, for example, a digital signal processor (DSP) or a hybrid analog and digital circuit, to provide the desired controller functions.

In operation, for example, the controller 260 causes the current regulation circuit 280 to operate in a first or second mode based on the magnitude of the received feedback signals I_{F} and V_{F}. In the first mode, alternatively referred to as the voltage control mode, the controller 260 generates control signal C₁ with a value to cause the current regulation circuit 280 to pass voltage V_{R} generated by the voltage regulation circuit 210 directly to the switching circuit 220 with little or no attenuation. In the second mode, alternatively referred to as the current control mode, the controller 260 generates the control signal C₁, with a value to cause current regulation. In this mode, the current regulator circuit 280 passes voltage V_{R} generated by the voltage regulation circuit 210 through impedance Z to the switching circuit 220, i.e., providing a corresponding reduced voltage to the switching circuit relative to the voltage provided when the current regulator 280 is operated in its first mode.

Suitable values for changes in V_{R} in this mode relative to the first mode are, for example, typically from between 0% and approximately 25% reduction in the voltage V_{R}. The particular change in V_{R} selected for this mode will be based upon, for example, nozzle geometry, formulation characteristics, and environmental conditions. During operation, the controller 260 monitors the feedback current signal I_{F}. If the signal I_{F} possesses a magnitude below a threshold value, then the control signal C₁ is produced to cause the switching circuit 220 to operate in its voltage control mode. If the monitored feedback current signal I_{F} reaches or exceeds the threshold value, then the control signal C₁, is generated to cause the switching circuit 220 to operate in its current regulated mode with an increased attenuation of the signal V_{R} based on a transfer function of the controller 260. The transfer function may be determined by empirical data. Suitable transfer functions useable with the invention include, for example, constant current, constant power, or a non-linear response or some combination thereof.

It is possible to refer to the first mode of operation as a constant voltage mode assuming that the voltage regulation circuit 210 provides a voltage to the current regulation circuit 280, and subsequently the switch circuit 220 and correspondingly the transformer 230 of substantially constant magnitude. In another embodiment, it is also possible to refer to the second mode of operation in which the current regulation circuit 280 is limiting the voltage signal V_{R} as a substantially constant power mode as the power provided to the transformer 230 would be substantially constant, i.e., V_{R}²/Z, if the voltage regulation circuit 210 provides a substantially constant voltage to the switch circuit 220. In other embodiments, there may be multiple operating modes or a single operating mode where the control signal C₁, is generated to adjust or regulate the voltage signal V_{R}

The switching circuit 220 provides a desired modified voltage signal based on voltage signal V_{R}. In some instances, the modified signal is similar to a square wave. The switching circuit 220 provides an “on-off” type signal to the transformer 230 in such a manner that the “time-average” of the on and off is equivalent to the voltage signal V_{R}, and the voltage signal V_{R} is correlated directly to the high voltage output V_{O} as controlled by the controller 260 and the current regulation circuit 280. It is desirable for the current regulation circuit 280 to minimize fluctuations of any given voltage so that V_{R} (and ultimately V_{O}) remain within a given tolerance range.

In the embodiment illustrated in FIG. 2, the feedback voltage signal V_{F} is not adjusted by the controller 260. Instead, the signal V_{F} is directly provided to voltage regulation circuit 210 to maintain its output relatively constant with a minimal variance, for example, about a 5% change, in output voltage Vᵢ of the voltage regulation circuit 210. It is desirable to maintain such output voltage of the voltage regulation circuit 210 within such tolerance range as it directly effects the tolerance of the desired goal of, for example, droplet size.

In FIG. 2, the controller 260 may also receive environmental information from an optional environmental sensor or sensors 270. Such sensors may, for example, measure temperature, humidity, and/or pressure. The corresponding environmental information received by the controller 260 may advantageously be used as input to the transfer function maintained by the controller 260.

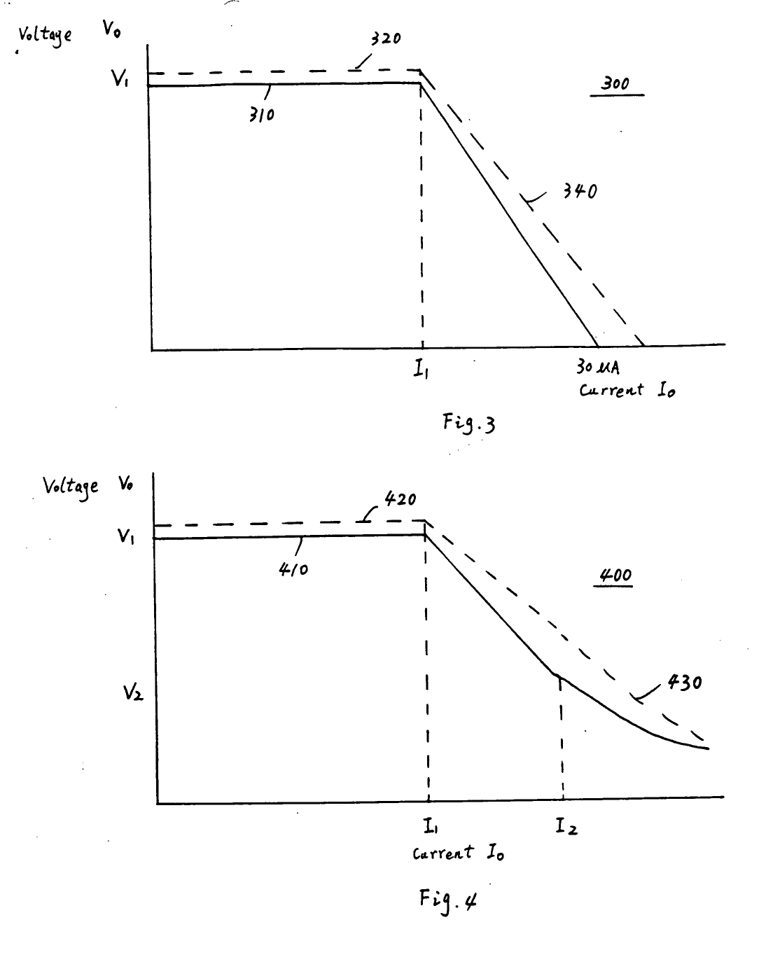

In operation, the exemplary power supply circuit 200 of FIG. 2 operates to regulate the provided voltage V_{O} and current I_{O} at the outputs 250 in accordance with the exemplary voltage current function plot 300 depicted in FIG. 3. Curve 310 of plot 300 is a voltage-current function that could be determined empirically as the relatively ideal or useable approximation of the operating conditions for achieving the desired EHD performance. Once the desired operating conditions are known as in curve 300, then a plot of the control function can be set and the transfer function determined. Thus, if the curve 310 is determined empirically, then the actual operating curve for the transfer function may be set to depicted curve 320. Note, it is desirable to have the curves 320 to superimpose or overlap with the curve 310. However, in FIG. 3, the curves 310 and 320 are not shown overlapping or superimposed for ease of illustration and explanation purposes only.

Accordingly, in the previously described exemplary embodiment in FIG. 2, it would be advantageous for the transfer function to maintain the control signal C₁ at magnitude to operate the circuit its voltage control mode until such time as the feedback current signal I_{F} is equal to value I₁ in FIG. 3 and then the control signal C₁, would increase linearly between the values I₁ and I₂, or alternatively until the output voltage Vₒ is equal to 0.

The operation of the power supply circuit 200 of FIG. 2 will now be described with respect to the output voltage and current graph 300 of FIG. 3. The voltage regulation circuit 210 generates a substantially constant voltage V_{R}, on the order of, for example, 2V that is provided to the switch circuit 220. Curve 310 has been empirically determined for a given device design and liquid formulation. It is based on attempting to optimize EHD efficiency, i.e., droplet size. If the produced droplets are too big, then they may not flow in the desired path, but instead be influenced substantially by inertial forces, such as gravity. If the droplets are too small again they may not reach their target.

Thus, the magnitude of the output voltage Vₒ is critical to EHD performance. If the output voltage Vₒ is below a threshold limit, then aerosolization will not occur. However, if the output voltage Vₒ is above the threshold limit, but not high enough, the resulting droplets will be too big. Likewise, if the output voltage Vₒ is too high above the threshold limit, then the droplets produced will also be too big. In other voltage regions, the droplets may be too small.

An exemplary method for determining a suitable voltage-current function curve useable for aerosolizing liquid by way of an electric field having a physical characteristic maintained in a desired range over varying operating conditions is to experimentally determine such function by testing and monitoring the physical properties during aerosolization of a liquid with different voltages, currents and frequencies over a varying range of the operating conditions. Once a suitable voltage-current (and/or frequency) function curve has been determined then a corresponding regulated power supply can be configured to approximate or accurately produce the determined voltage-current function for generating the electric field.

Referring again to FIG. 2, initially, using the control signal C₁, the controller 260 controls the current regulation circuit 280 to operate in its first mode of operation so that the voltage V_{R} is applied to the switch circuit 220 which then feeds a corresponding voltage to the transformer 230 which then provides a corresponding stepped up voltage to the high voltage multiplier stages 240 which generates an even higher voltage V_{O} at its output. As shown in the function region 310 of FIG. 3, the resulting output voltage V_{O} will be at voltage V₁. Suitable voltage values for voltage V₁, are on the order of, for example, 10 KV to 12 KV with the current drawn being less than current I₁, for generating an electric field for aerosolizing liquid. The current I₁ can be on the order of, for example, 10 82 A.

Feedback voltage and current signals V_{F} and I_{F} produced by the high voltage stages circuit 240 are provided to the voltage regulation circuit 210 and the controller 260, respectively, with an indication of the corresponding values of the output voltage and current V_{O} and I_{O}. The drawn output current I_{O} is dependent upon the effective impedance of the issuing liquid in combination with environmental conditions such as, for example, relative humidity, temperature, proximate distances between electrodes, the volume of fluid passing through the electric field, which may also be effected by variations in the nozzle tip diameter. If the controller 260 detects that drawn output current I_{O} is larger than current I₁ as depicted in FIG. 3 then it controls the current regulation circuit 280 in FIG. 2 via the control signal C₁ to switch to its second mode of operation and reduces its output voltage and an optimized voltage to the switch circuit 220 and subsequently to the transformer 230 which likewise reduces its output voltage and provides a substantially constant power to the high voltage multiplier stages 240 which has the same corresponding effect on the output voltage V_{O}. The reduced output voltage V_{O} is depicted as the linear slope 340 portion of curve 320.

As was previously stated, such reduction of voltage V_{O} in view of elevated output current I_{O} has the effect of maintaining a physical characteristic of the aerosolized liquid such as, for example, droplet size to be consistently within the range of, for example, 0.1 to 6 microns for therapeutic liquids. In exemplary embodiments of the invention it is advantageous for I_{O} to vary in a range by ±3 to ±4 μA.

Although FIG. 3 depicts the empirically determined voltage-current function curve 310 and transfer function voltage-current function curve 320 as different curves for ease of discussion and illustration purposes only. It should be readily understood that it is possible to employ identical curves for the empirically determined voltage-current function and corresponding implemented transfer function voltage-current function in a power supply circuit in accordance with the invention.

It is further possible to employ the optional environmental sensor 270 to better anticipate the desired output voltage V_{O}. The exemplary power supply configuration 200 was depicted in FIG. 2 for ease of illustration and it should readily be understood that numerous alternative configurations are useable with the present invention for providing a regulated output voltage and current function depicted in FIG. 3 to produce an aerosolized liquid having a substantially consistent desired physical characteristic over a broad range of environmental conditions.

FIG. 4 depicts an output voltage current graph 400 that illustrates a circuit performance that is useable to extend operation of the device 10 of FIG. 1 over an even broader range of environmental conditions than as described with respect to the output voltage and current graph 300 of FIG. 3. FIG. 4 depicts an empirically determined voltage-current function curve 410 and the corresponding actual voltage current function curve 420 used for determining the circuit transfer function that is more complex than that depicted on FIG. 3. In accordance with the curve 420, it is possible, in accordance with one embodiment of the invention, to add additional circuitry to the current regulation circuit 280 of FIG. 2 for providing an optional third mode of operation over that described relative to FIG. 3. It should be readily understood by one skilled in the art that there are many different analog or digital circuit configurations for use as the current regulation circuit 280 for providing this third mode function. In the alternative, it is possible to employ a current regulation circuit 280 without any additional circuitry if the control 280 is capable of controlling such current regulation circuit to produce the desired third mode function operation.

The circuitry for performing this third mode of operation should provide a sufficient non-linear response so as to cause output voltage V_{O} to track the voltage-current function curve 420 depicted in FIG. 4 in region 430 when the drawn current is larger than current I₂. A suitable value for current I₂ is on the order of, for example, 15 μA.

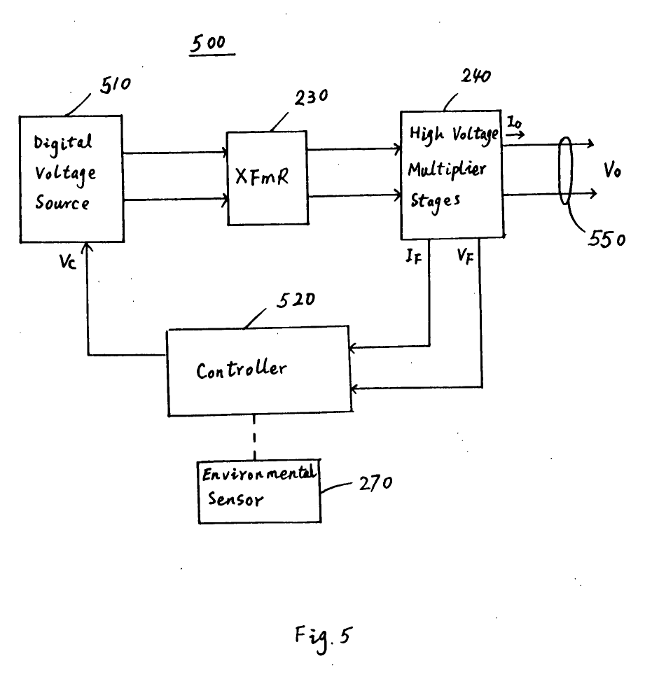

The design and configuration of the exemplary power supply circuit 200 of FIG. 2 having two or optionally three modes was to approximate a desired voltage-current function curve 310 and 410 of FIGS. 3 and 4. It is alternatively possible to employ an increased number of operation modes in a regulated power supply circuit to more accurately track a desired voltage-current function curve. Moreover, it is further possible to employ a digital power supply and control unit to provide such operational modes or to employ a single mode that accurately tracks a desired voltage-current function curve. An exemplary digital regulated power supply circuit 500 useable for such purpose is depicted in FIG. 5. A digital regulated power supply circuit makes it possible to implement multiple transfer functions or emulate different circuits. For example, rather than an impedance based circuit in the current regulation circuit, it would be possible to employ a set of different resistors that are switched into the circuit as the feedback current signal I_{F} changes.

The power supply circuit 500 in FIG. 5 is similar to the power supply circuit 200 in FIG. 2 and employs like transformers 230 and high voltage multiplier stages 240 and optional environmental sensor 270. However, the voltage regulation circuit 210 and current regulation circuit 280 of FIG. 2 have been substituted by a digital voltage source 510 in the circuit 500 of FIG. 5. In another embodiment, the switching circuit could also be part of the digital voltage source. In a similar manner, the controller 520 in FIG. 5 replaces the controller 260 of FIG. 2.

In operation of the power supply circuit 500 of FIG. 5, the controller 520, which may be, for example, one or more digital signal processors, provides control signal V_{C} to adjust the voltage from source 510 which is amplified by the transformer 230 and high voltage multiplier stages 240 to produce output voltage V_{O} and current I_{O} of the desired magnitudes in accordance with a desired voltage current function to compensate for differing environmental conditions.

The configuration of the depicted power supply circuits 200 and 500 in FIGS. 2 and 5 are for illustration purposes only and it should be readily understood that a large number of different circuit configurations may be employed to produce the desired output voltage and current V_{O} and I_{O} relationship in accordance with the invention. For example, the transformer 230 and/or high voltage multiplier stages 240 may be omitted if the voltage regulation circuit 210 and digital voltage source 510 alone, or in combination with other components, provide the necessary high voltage for generating the aerosolization electric field. It is alternatively possible to employ a piezoelectric transformer for producing the required voltage.

The embodiments of the invention previously described with regard to FIGS. 2 through 5 employ determined transfer functions to adjust the output voltage V_{O} based on changes in the operating conditions by monitoring the magnitude of the output current I_{O} alone or in combination with measurements by the environmental sensors 270 in FIGS. 2 and 5. However, in accordance with another exemplary embodiment of the invention, it is possible for the controllers 260 and 520 in FIGS. 2 and 5 to adjust the output voltage V_{O} based on only measurements from the environmental sensors 270. It is alternatively possible in such embodiments to eliminate the feedback current signal I_{F} as an input to the controller 260 or 520 in FIGS. 2 and 5.

It should be understood that, although liquid spray embodiments of the invention are shown and described herein with regard to an inhalation device, embodiments of the invention are suitable for use in spraying crops, paint or for liquids intended to cover a surface. For instance, the invention has been described as a single voltage EHD device, i.e., with one or more electrodes, such as the nozzle electrode maintained at ground while other electrodes are charged to the desired voltage, for ease of discussion purposes only. The invention is also applicable to EHD devices that employ electrodes charged to two or more different voltages. In such instances, it is possible to employ two or more corresponding control circuits in accordance with the invention. It will be apparent to those skilled in the art that many other changes and substitutions can be made to the power supply circuit configuration or electric field generator described herein without departing from the spirit and scope of the invention as defined by the appended claims and their full scope of equivalents.

The invention claimed is:

1. A device for dispensing an aerosolized liquid comprising:
a liquid supplier having a liquid outlet;
an electric field generator for generating an electric field to cause liquid issuing from the liquid outlet to be aerosolized for dispensing; and
a power supply electrically coupled to the electric field generator, said power supply for regulating an electrical characteristic of its output based on detected operating conditions of said device.

2. The dispensing device of claim 1 wherein said power supply output regulation is for causing an aerosolized liquid having a particular characteristic when said device is subject to a range of differing environmental conditions during operation.

3. The dispensing device of claim 1 wherein said detected operating conditions corresponds to signals received from at least one environmental sensor.

4. The dispensing device of claim 1 wherein said detected operating conditions corresponds to a detected electrical characteristic in generating said electric field.

5. The dispensing device of claim 4 wherein said detected electrical characteristic of said electric field generation corresponds to a current drawn by said electric field generator.

6. The dispensing device of claim 1 wherein said power supply is adapted to operate in at least two modes based on said current drawn, a first mode whereby said regulated electrical characteristic is a substantially constant voltage provided to said electric field generator and a second mode whereby said regulated electrical characteristic is a substantially constant power provided to said electric field generator.

7. The dispensing device of claim 6 wherein said power supply is further adapted to operate in said first mode when said current drawn is within a first range and in said second mode when said current drawn is within a second range.

8. The dispensing device of claim 7 wherein the power supply is further adapted to operate in a third mode when said current drawn is in a third range.

9. The dispensing device of claim 8 wherein said third mode provides a non-linear voltage current function.

10. The dispensing device of claim 1 wherein said regulated electrical characteristic is a voltage provided to said electric field generator.

11. The dispensing device of claim 1 wherein said electric field generator comprises:
an electrically conductive nozzle coupled to said liquid outlet; and
an electrode disposed proximate and in relation to said electrically conductive nozzle for creating said electric field.

12. The dispensing device of claim 1 wherein said device is an inhaler.

13. The dispensing device of claim 1 wherein said liquid includes a medicament.

14. The dispensing device of claim 1 wherein said electric field generator comprises at least two electrodes disposed within a nozzle coupled to said liquid outlet.

15. The dispensing device of claim 1 wherein said device is adapted to dispense such aerosolized liquid over an intended surface area.

16. The dispensing device of claim 15 wherein said liquid includes an insecticide or a biocide.

17. The dispensing device of claim 15 wherein said liquid includes a nutrient.

18. The dispensing device of claim 15 wherein said liquid includes a paint.

19. A method for dispensing an aerosolized liquid comprising the steps of:
generating an electric field proximate an outlet of a liquid supplier to cause liquid issuing from said liquid outlet to be aerosolized for dispensing;
detecting an operating condition; and
regulating an electrical characteristic of a power supply output used for said electric field generation based on said detected operating condition.

20. The method of claim 19 wherein said regulated power supply electrical characteristic is voltage to maintain a desired physical characteristic of said dispensed liquid when said method is performed subject to a range of environmental conditions.

21. The method of claim 19 wherein said detecting step further comprises the step of receiving a signal from at least one environmental sensor.

22. The method of claim 19 wherein said detecting step further comprises the step of detecting electrical characteristic of said electric field generation.

23. The method of claim 22 wherein said detected electrical characteristic of said generated electric field corresponds to a current drawn by said electric field generation.

24. The method of claim 23 wherein the regulation step further comprise the step of regulating voltage as said regulated power supply electrical characteristic in a first or second mode based on said detected drawn current.

25. The method of claim 24 wherein the first mode provides a substantially constant voltage for generation of said electric field and the second mode provides a substantial constant power for generation of said electric field.

26. The method of claim 24 wherein the voltage regulation step further operates in the first mode when said current drawn is within a first range and in said second mode when said current draw is within a second range.

27. The method of claim 26 wherein the voltage regulation step further operates in the third mode when said detected drawn current is in a third range.

28. The method of claim 27 wherein said third mode corresponds to a non-linear voltage current function.

29. The method of claim 19 wherein said electric field generation step further comprises the steps of:
coupling an electrically conductive nozzle to said liquid outlet; and
positioning an electrode proximate and in relation to said electrically conductive nozzle for creating said electric field.

30. The method of claim 19 wherein said electric field generation step further comprises the step of disposing electrodes with a nozzle coupled to said liquid outlet.

31. The method of claim 19 further comprising the step of dispensing the aerosolized liquid in a manner suitable for inhalation by a patient.

32. The method of claim 31 wherein said liquid includes a medicament.

33. The method of claim 19 further comprising the step of dispensing the aerosolized liquid in such a manner as to be applied over an intended surface area.

34. The method of claim 33 wherein said liquid includes a biocide or insecticide.

35. The method of claim 33 wherein said liquid includes a nutrient.

36. The method of claim 33 wherein said liquid includes a paint.

37. A method for dispensing an aerosolized liquid comprising the steps of:
generating an electric field proximate an outlet of a liquid supplier to cause liquid issuing from said liquid outlet to be aerosolized for dispensing; and
regulating a voltage of a power supply output used for said electric field generation, said voltage regulation based on a transfer function and a current drawn by said electric field generation.

38. The method of claim 37 wherein said transfer function is empirically determined.
